(19) United States
(12) Patent Application Publication
(10) Pub. No.: US 2025/0248632 A1
(43) Pub. Date: Aug. 7, 2025

(54) MULTIMODE SENSOR INTEGRATION ONTO NEEDLE-BASED SENSING SYSTEMS

(71) Applicant: University of Cincinnati, Cincinnati, OH (US)

(72) Inventors: Jason Charles Heikenfeld, Cincinnati, OH (US); Zachary Lee Watkins, Cincinnati, OH (US)

(21) Appl. No.: 18/856,645

(22) PCT Filed: Apr. 19, 2023
(86) PCT No.: PCT/US2023/019155

§ 371 Date: Oct. 14, 2024

Related U.S. Application Data
(60) Provisional application No. 63/359,547, filed on Jul. 8, 2022.
(60) Provisional application No. 63/332,512, filed on Apr. 19, 2022.

(51) Int. Cl. A61B 5/1486; A61B 5/145

(52) U.S. Cl. CPC A61B 5/14865; A61B 5/14532; A61B 5/14546; A61B 2562/063

(57) ABSTRACT

A wearable device for sensing at least one analyte in a biofluid is provided. The wearable device includes at least one feature configured to penetrate a skin of a use. The wearable device further includes a first sensor coupled to the feature, the first sensor configured to detect a first analyte. wherein the first sensor is in fluid communication with the feature. The wearable device further includes a second sensor configured to detect a second analyte. the second analyte being an analyte different from the first analyte. wherein the second analyte is in fluid communication with the feature.

### CROSS REFERENCE TO RELATED APPLICATIONS

The present application is an International Patent Application which claims priority to, and the benefit of the filing date of, U.S. Ser. No. 63/359,547, titled “Multimode Sensor Integration onto Needle-Based Sensing Systems”, which was filed Jul. 8, 2022 the disclosure of which is hereby incorporated herein by reference in its entirety. The present application also claims priority to, and the benefit of the filing date of U.S. Ser. No. 63/332,512, titled “Multimode Sensor Integration onto Needle-Based Sensing Systems” which was filed Apr. 19, 2022 the disclosure of which is hereby incorporated herein by reference in its entirety.

### FIELD OF THE INVENTION

This invention relates generally to the integration of aptamer sensors onto platforms that use an additional distinct sensor modality such as enzymatic sensors.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Electrochemical aptamer sensors can identify the presence and/or concentration of an analyte of interest via the use of an aptamer sequence that specifically binds to the analyte of interest. These sensors include aptamers attached to an electrode, wherein each of the aptamers has a redox active molecule (redox tag) attached thereto. The redox couple can transfer electrical charge to or from the electrode. When an analyte binds to the aptamer, the aptamer changes shape, bringing the redox couple closer to or further from, on average, the electrode. This results in a measurable change in electrical current that can be translated to a measure of concentration of the analyte. Aptamers are an example of an affinity-based biosensor.

A major unresolved challenge for aptamer sensors and other affinity-based biosensors (particularly those where the aptamers are bonded to the working electrode) is introducing them into the commercial market in an impactful manner. In part, commercial introduction has been hampered by a lack of sensor longevity. Another significant limitation is stand-alone utility of an aptamer sensor. Regarding this second limitation, it is easier to introduce an additional sensor onto a platform if the platform is one that is already being used, such that the additional cost or burden to the user is minimal. For example, diabetics already use continuous glucose meters and adding an aptamer sensor to a continuous glucose meter may have higher commercial feasibility than introducing an aptamer sensor alone into the market. However, integration of multiple sensors onto a platform is challenging, especially if the sensing modalities are different. For example, an enzymatic sensor can be applied by simple lamination or dip-coating methods, whereas conventional aptamer sensors are monolayer-layer based and therefore require solution-phase deposition where a monolayer of aptamer and blocking layer is ‘incubated’ onto an electrode surface such as gold. These processes are incompatible with each other. What are needed are novel methods which resolve these bottlenecks to sensor integration.

### SUMMARY OF THE INVENTION

Aspects of the disclosed invention are directed to multimode sensors integrated onto a feature to form a sensing system. Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with biofluid and analytes.

One aspect of the present invention is directed to a device for sensing at least one analyte in a biofluid, comprising at least one needle-shaped element. The needle shaped feature has attached thereto a first sensor for a first analyte and a second sensor for a second analyte.

For example, in an embodiment, a wearable device for sensing at least one analyte in a biofluid is provided. The wearable device includes at least one feature configured to penetrate a skin of a user. The wearable device further includes a first sensor coupled to the feature, the first sensor configured to detect a first analyte, wherein the first sensor is in fluid communication with the feature. The wearable device further includes a second sensor comprising a plurality of aptamers configured with a plurality of aptamers to detect a second analyte, the second analyte being an analyte different from the first analyte, wherein the second analyte is in fluid communication with the feature.

In a related embodiment, the wearable device further includes the feature including a needle.

In a related embodiments, the wearable device further includes the feature including film, gold or stainless-steel.

In a related embodiments, the wearable device further includes at least one of the first sensor or the second sensor being an enzymatic sensor.

In a related embodiments, the wearable device further includes the first analyte being glucose.

In a related embodiments, the wearable device further includes at least one of the first sensor or the second sensor being an aptamer-based sensor.

In a related embodiments, the wearable device further includes the first sensor being a continuous monitoring sensor configured to measure the first analyte during a use of the wearable device.

In a related embodiments, the wearable device further includes the second sensor being a continuous monitoring sensor configured to measure the second analyte during the use of the wearable device.

In a related embodiments, the wearable device further includes the second sensor being a non-continuous monitoring sensor configured to measure the second analyte during only a portion of the use of the wearable device.

In a related embodiments, the wearable device further includes the second sensor being configured to provide a single concentration reading of the second analyte.

In a related embodiments, the wearable device further includes the second analyte being a drug.

In a related embodiments, the wearable device further includes a third sensor wherein the third sensor is configured to detect an analyte.

In a related embodiments, the wearable device further includes the second analyte being a biomarker analyte.

In a related embodiments, the wearable device further includes a third sensor wherein the third sensor is configured to detect a second biomarker analyte that is not the same biomarker analyte that is configured to be detected by the second sensor.

In a related embodiments, the wearable device further includes at least one of the first analyte or the second analyte being a blood thinner drug.

In a related embodiments, the wearable device further includes at least one of the first analyte or the second analyte being a c-reactive protein.

In a related embodiments, the wearable device further includes at least one of the first analyte or the second analyte being a troponin.

In a related embodiments, the wearable device further includes at least one of the first analyte or the second analyte being a brain-natriuretic peptide.

In a related embodiments, the wearable device further includes at least one of the first analyte or the second analyte being insulin.

In a related embodiments, the wearable device further includes the first sensor being an enzymatic sensor and is covered by a first membrane, and the second sensor is an aptamer sensor, and the first and the second sensors are both further covered by a second membrane that is at least 5× more permeable to glucose than the first membrane.

A method of determining an initial concentration of a first analyte and a second analyte in a sample fluid is also provided. The method includes providing a first sensor including a plurality of enzymes, the plurality enzymes each including at least one binding site configured to react to the first analyte. The method further includes providing a second sensor including a plurality of aptamers, the plurality of aptamers each including at least one binding site configured to bind to the second analyte. The method further includes providing the sample fluid including the first analyte and the second analyte. The method further includes introducing the sample fluid to the plurality of plurality of enzymes and the plurality or aptamers to allow binding of the first analyte to at least one of the plurality of enzymes and allow binding of the second analyte to at least one of the plurality of aptamers, wherein the first analyte reacting the to at least one of the plurality of enzymes produces a change in a first signal having a first signal strength and the second analyte binding to the at least one of the plurality of aptamers produces a second change in a second signal having a second signal strength. The method further includes depleting the first analyte from being unbound to at least one of the plurality of enzymes to the extent that the first signal strength is unchanged for a period of time. The method further includes depleting the second analyte from being unbound to at least one of the plurality of aptamers to the extent that the second signal strength is unchanged for a period of time. The method further includes calculating the initial concentration of the first analyte in the sample fluid based on the first signal strength after depleting the first analyte from being unreacted to at least one of the plurality of enzymes to the extent that the first signal strength is unchanged for the period of time. The method further includes calculating the initial concentration of the second analyte in the sample fluid based on the second signal strength after depleting the second analyte from being unbound to at least one of the plurality of aptamers to the extent that the second signal strength is unchanged for the period of time. A wearable device for sensing at least one analyte in a biofluid is also provided. The wearable device includes at least one feature configured to penetrate a skin of a user. The wearable device further includes a sensor coupled to the feature, the sensor configured to detect the at least one analyte, wherein the sensor is in fluid communication with the feature. The wearable device further includes a sacrificial layer housing the sensor, the sacrificial layer being removable from the wearable device in response to at least one of an application of a predetermined amount of heat or solution to dissolve the sacrificial layer.

### BRIEF DESCRIPTION OF THE DRAWINGS

The objects and advantages of the disclosed invention will be further appreciated in light of the following detailed descriptions and drawings in which:

FIG. 1 is a schematic of a conventional prior art sensor device.

FIG. 2A is a schematic of a device with two or more sensors in an embodiment of the present invention.

FIG. 2B is a schematic of a portion of a device with two or more sensors in an embodiment of the invention.

FIG. 3 is a schematic of a portion of a device with two or more sensors in an embodiment of the present invention.

FIG. 4 is a schematic of a device with two or more sensors in an embodiment of the present invention.

FIG. 5 is a schematic of a device with two or more sensors in an embodiment of the present invention.

FIG. 6A is a schematic of an alternate embodiment of the present invention showing a device with a sensor housed in a sacrificial layer.

FIG. 6B is a schematic of an alternate embodiment of the present invention showing a device with a sensor previously housed in a sacrificial layer wherein the sacrificial material has been removed.

FIG. 6C is a schematic of an alternate embodiment of the present invention showing a device with two or more sensors.

FIG. 6D is a schematic of an alternate embodiment of the present invention showing a device with two or more sensors.

FIG. 7A is a view of a portion of a device described herein.

FIG. 7B is a view of a portion of a device described herein.

FIG. 8 is a view of a portion of a device described herein.

### DEFINITIONS

As used herein, “continuous sensing” with a “continuous sensor” means a sensor that changes in response to changing concentration of at least one solute in a solution such as an analyte. Similarly, as used herein, “continuous monitoring” means the capability of a device to provide multiple measurements of an analyte over time.

As used herein, the term “about,” when referring to a value or to an amount of mass, weight, time, volume, pH, size, concentration or percentage is meant to encompass variations of ±20% in some embodiments, ±10% in some embodiments, ±5% in some embodiments, ±1% in some embodiments, ±0.5% in some embodiments, and ±0.1% in some embodiments from the specified amount, as such variations are appropriate to perform the disclosed method.

As used herein, the term “electrode” means any material that is electrically conductive such as gold, platinum, nickel, silicon, conductive liquid infused materials such as ionic liquids, PEDOT:PSS, conductive oxides, carbon, boron-doped diamond, nanotubes or nanowire meshes, or other suitable electrically conducting materials.

As used herein, the term “blocking layer” means a homogeneous or heterogeneous layer of material or of one or more types of molecules on an electrode which reduce electrochemical background current and/or current due to electrochemical interference, and which may promote proper freedom of movement for the aptamer which is required for creating a measurable response to analyte concentration.

As used herein, the term “antifouling layer” means a homogeneous or heterogeneous layer of material or of one or more types of molecules on a surface which reduces fouling on a surface compared to if such an antifouling layer was not utilized. A blocking layer may also act as an antifouling layer.

As used herein, the term “aptamer” means a molecule that undergoes a conformation or binding change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function, but which behave analogous to traditional aptamers. Two or more aptamers bound together can also be referred to as an aptamer (i.e., not separated in solution). Aptamers can have molecular weights of at least 1 kDa, 10 kDa, or 100 kDa.

As used herein, the term “redox tag” or “redox molecule” means any species such as small or large molecules with a redox active portion that when brought adjacent to an electrode can reversibly transfer at least one electron with the electrode. Redox tag or molecule examples include methylene blue, ferrocene, quinones, or other suitable species that satisfy the definition of a redox tag or molecule. In some cases, a redox tag or molecule is referred to as a redox mediator. Redox tags or molecules may also exchange electrons or change in behavior when brought into proximity with other redox tags or molecules. Exogenous redox molecules are those added to a device, e.g. they are not endogenous and provided by the sample fluid to be tested.

As used herein, the term “change in electron transfer” means a redox molecule whose electron transfer with an electrode has changed in a measurable manner. This change in electron transfer can, for example, originate from availability for electron transfer, distance from an electrode, diffusion rate to or from an electrode, a shift or increase or decrease in electrochemical activity of the redox molecule, or any other embodiment as taught herein that results in a measurable change in electron transfer between the redox molecule and the electrode.

As used herein, the term “sensing monolayer” means at least a plurality of aptamers on a working electrode, which may also include a plurality of molecules or mixtures of molecules that form a blocking layer and/or an anti-fouling layer.

As used herein, the term “analyte” means any solute in a solution or fluid which can be measured using a sensor. Analytes can be small molecules, proteins, peptides, electrolytes, acids, bases, antibodies, molecules with small molecules bound to them, DNA, RNA, drugs, chemicals, pollutants, or other solutes in a solution or fluid.

As used herein, the term “continuous sensing” simply means the device records a plurality of readings over time. Even a point-of-care testing device which provides a single data point can be considered a continuous sensing device if, for example, it is a 15 minute test, that operates by taking multiple data points over 15 minutes and averaging them to provide a single data measure.

As used herein, a “device” comprises at least one sensor based on at least one aptamer, at least one sensor solution, and at least one sample solution. Devices can sense multiple samples and be in multiple configurations such as a device to measure a pin-prick of blood, or a microneedle or in-dwelling sensor needle to measure interstitial fluid, or a device to measure saliva, tears, sweat, or urine sensor, or a device to measure water pollutants or food processing solutes, or other devices which measure at least one analyte found in a sample solution.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a reference or counter electrode, a battery, antenna, adhesive), and for purposes of brevity and focus on inventive aspects, such components may not be explicitly shown in the diagrams or described in the embodiments of the disclosed invention. All ranges of parameters disclosed herein include the endpoints of the ranges.

With reference to FIG. 1, a conventional prior art sensor device 100 is shown placed initially in a sample fluid 130 such as dermal interstitial fluid of skin 12 is shown, comprising: at least one working electrode, such as sensor 120, which may be made of a material such as gold, carbon, or other suitable electrode material. Counter and references electrodes are not shown, but may be included on feature 170 and or as part of adhesive 110. The device also comprises electronics 160 for reading the sensor 120 and communicating data to a user or smart phone (not shown). The feature 170 extends from the electronics 160 and may include a needle or other arm that is configured to penetrate the skin 12 of a user when the device 100 is used. The feature 170 includes an end coupled to the electronics 160 and an end having a tip 150 opposite the end coupled to the electronics 160. Sensor 120 may be an enzymatic glucose sensor based on glucose oxidase. Alternatively or in addition, sensor 120 may also be an aptamer sensor comprising at least one blocking layer a plurality of molecules such as mercaptohexanol that are thiol bonded to the electrode, and at least one aptamer that is responsive to binding to an analyte and which contains a redox tag such as methylene blue. Further aptamer examples will be taught in later examples.

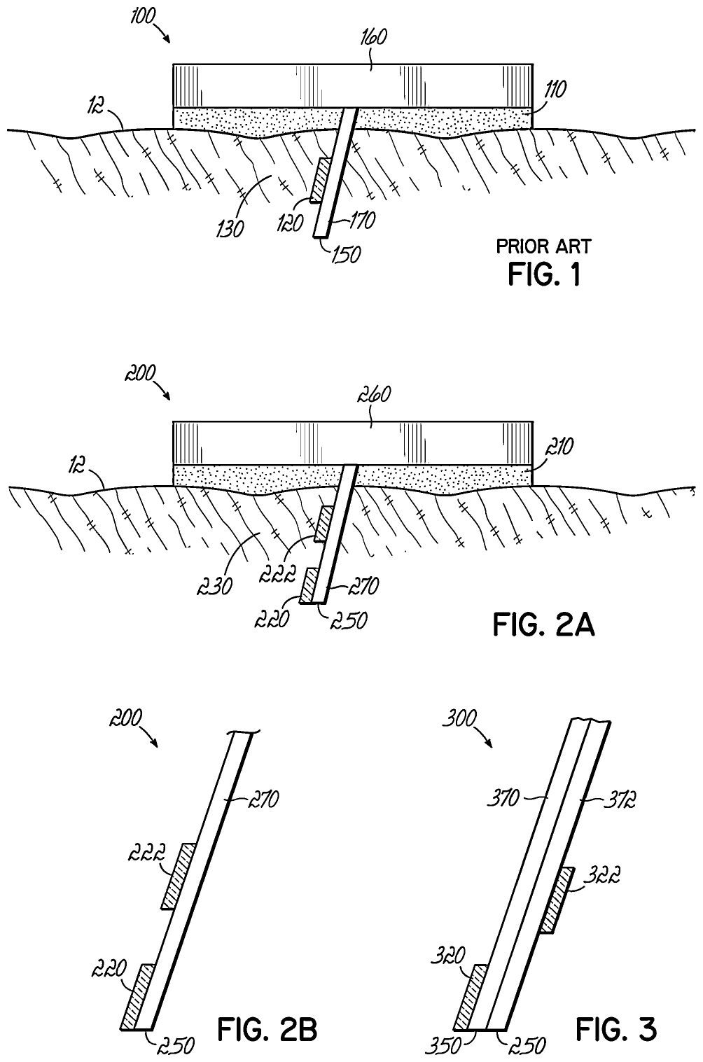

With reference to FIG. 2A, where like numerals refer to like features, in an embodiment of the present invention a device 200 includes a single feature 270, such as but not limited to the needle element shown in FIG. 2. The single feature 270 may be made of materials such as a Kapton film, gold, tantalum, or stainless-steel, which further carries a first sensor 220 for a first analyte and a second sensor 222 for a second analyte. For example, the first sensor 220 may be an enzymatic sensor for glucose while the second sensor 222 may be an aptamer sensor for a drug. In some examples, the second sensor 222 is configured to detect analytes such as the blood thinner warfarin or rivaroxaban, detection of which may be indicative of cardiovascular complications which are comorbidities of diabetes. Alternately or in addition, second sensor 222 may be configured to detect insulin which is directly related to diabetes. Alternately second sensor 222 may be configured to detect C-reactive protein which is directly related to diabetes and/or its complications. Embodiments of the present invention may therefore include at least a first sensor 220, such as a sensor for glucose, and at least one additional sensor, such as second sensor 222, which, as described above may include an aptamer sensor, carried on a common feature 270, such as a needle as shown in FIG. 2. Embodiments of the invention may therefore include at least one sensor for diabetes and at least one additional sensor for a comorbidity of diabetes. Furthermore, the device 200 may include electronics 260 for reading the first sensor 220 and second sensor 222 and communicating data to a user or smart phone (not shown). The feature 270 extends from the electronics 260 and may include a needle or other arm that is configured to penetrate the skin 12 of a user when the device 200 is used. The feature 270 includes an end coupled to the electronics 260 and an end having a tip 250 opposite the end coupled to the electronics 260. Comorbidities may also include renal disease, chronic inflammation, and/or other health issues. While first sensor 220 is shown in FIG. 2A as further from the electronics 260 on the feature 270 than the second sensor 222, in another embodiment, the first sensor 220 may be located closer to the electronics 260 on the feature 270 than the second sensor 222.

With reference to FIG. 2B, a view of the feature 270 with the sensors 220, 222 coupled thereto is shown. In an embodiment of the present invention the first sensor 220 may be fabricated using lamination or dip coating methods as known by those skilled in the art of enzymatic sensors, and the second sensor 222, which may be an aptamer sensor, may be formed by solution-based methods by incubating an electrode such as gold associated with second sensor 222 into a solution of first aptamers for thiol bonding to the gold followed by a solution of mercaptohexanol or mercaptooctanol which together form a complete sensor. To ensure the sensor fabrication processes do not interfere with each other, the second sensor 222 end of feature 270, which may be a needle, may be dipped downward in the direction of gravity into an aptamer or blocking layer solution for fabricating the second sensor 222. Alternatively or in addition, a wicking material, sponge material, or capillary containing aptamer or blocking layer solutions can be placed against the location of electrode sensor 222, and moved at least once during positioning to ensure that no surface coverage is blocked, or just held in close proximity such that only solution touches the electrode surface to be functionalized. Though only two sensors 220, 222 are shown in FIG. 2A and 2B, a third sensor (or any additional number of sensors) could be added to the feature 270 as well using one or more methods such as the method taught for the shown sensors 220, 222.

With reference to FIG. 3, a view of the features 370, 372 with sensors 320, 322 coupled thereto is shown. In FIG. 3, like numerals refer to like features, and in an embodiment of the invention sensors 320, 322 are separately fabricated on two features 370, 372 such as needles or other suitable substrate materials. The features 370, 372 may be laminated or adhered together using methods such as ultrasonic welding, capillary wicking of a glue or epoxy, a thin film double sided adhesive, feature 370 being Kapton and feature 372 being adhesive backed Kapton, or other suitable methods. The feature 370 and the feature 372 may be made of the same or different materials, and are each configured to penetrate a user's skin 12, thus positioning the sensors 320, 322 in a desired location. While first sensor 320 is shown in FIG. 3 as nearer the tip 350 of feature 370 than the second sensor 322. In another embodiment, the first sensor 320 may be located further from the tip of the feature 370 than the second sensor 322. In an embodiment of the invention as shown in FIG. 3, the first sensor 320 is positioned on one feature 370 and the second sensor 322 is positioned on another feature 372. Furthermore, as shown in FIG. 3, the feature 370 has a tip 350 at one end and feature 372 has a tip 352 at one end. As shown in FIG. 3, the tips 350, 352 of the features 370, 372 are coplanar. Though only two sensors 320, 322 are shown in FIG. 3, a third sensor (or any number of additional sensors) could be added as well using one or more methods such as the method taught for the shown sensors 320, 322.

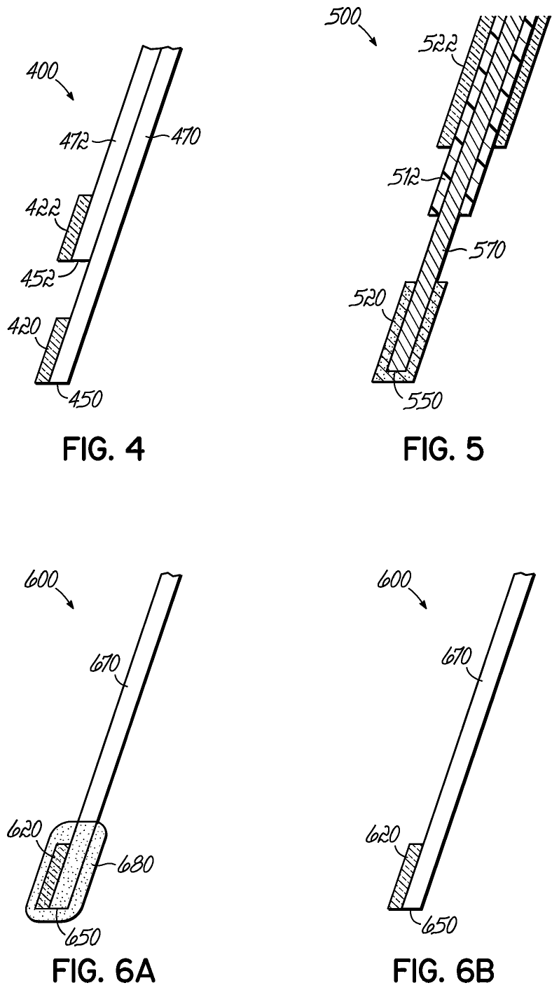

With reference to FIG. 4, where like numerals refer to like features, in an embodiment of the invention sensors are separately fabricated on two features 470, 472, such as needles or other suitable substrates. The features 470, 472 are laminated or adhered together using methods such as ultrasonic welding, capillary wicking of a glue or epoxy, a thin film double sided adhesive, or other suitable methods. The feature 470 and the feature 472 may be made of the same or different materials, and are each configured to penetrate a user's skin 12, thus positioning the sensors 420, 422 in a desired location. As shown in FIG. 4, each of the sensors 420, 422 positioned on its respective feature 470, 472 is positioned at the tip 450, 452. While first sensor 420 is shown in FIG. 4 as nearer the tip 450 of feature 470 than the second sensor 422, in another embodiment, the first sensor 420 may be located further from the tip of the feature 470 than the second sensor 422. In an embodiment of the invention as shown in FIG. 4, the first sensor 420 is positioned on one feature 470 and the second sensor 422 is positioned on another feature 472. Furthermore, as shown in FIG. 4, the feature 470 has a tip 450 at one end and feature 472 has a tip 452 at one end. As shown in FIG. 4, the tips 450, 452 of the features 470, 472 are not coplanar. Though only two sensors 420, 422 are shown in FIG. 4, a third or more sensor could be added as well using one or more methods such as the method taught for the shown sensors 420, 422.

With reference to FIG. 5, where like numerals refer to like features, in an embodiment of the invention sensors 520, 522 are separately fabricated on a single feature 570. In the embodiment shown in FIG. 5, the feature 570 is electrically conductive, and an insulating coating or insulating layer 512, referred herein as the insulating layer 512, is situated at least partially housing the feature 570. The insulating layer 512 may be made from materials such as polyamide, polyimide, urethane, acrylic, or other suitable polymer or electrically insulating material. A sensor 522 may be coupled onto the insulating layer 512 and another sensor 520 may be coupled directly onto feature 570. For example, a tantalum or stainless steel wire can be gold coated by vapor deposition or electroplating to form the feature 570, then a glucose sensor 520 fabricated using enzymatic chemistry and protective membrane chemistry may be coupled to the feature 570. The feature 570 includes an end coupled to the electronics (not shown) and an end having a tip 550 opposite the end coupled to the electronics. Before or after sensor 520 is coupled to the feature 570, insulating layer 512 may be applied by dip coating one end of the feature 570, and insulating layer 512 may be further coated with gold or other metal by shadow mask vacuum deposition. Feature 570 may be a film or a wire, and in the case of a wire, coatings are coaxial in nature. In an embodiment, the sensor 520 is coupled to the feature 570 at the tip 550, and the sensor 520 defines a space that houses a portion of the feature 570 including the tip 550. Though only two sensors 520, 522 are shown in FIG. 5, a third sensor (or any additional number of sensors) could be added as well using one or coatings as described herein (creating a multilayer coaxial set of sensors).

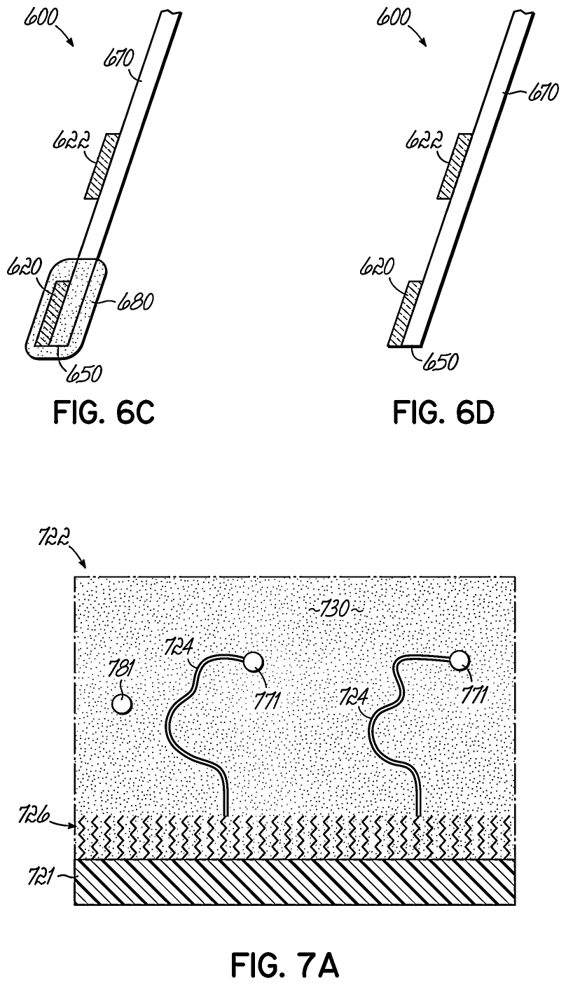

With reference to FIG. 6A, where like numerals refer to like features, a device 600 may include a sensor 620, which can be fabricated and coupled to a feature 670, such as a needle. The device 600 may further include a sacrificial protecting layer 680. The sensor 620 may be coupled to the feature 670 along the length of the feature 670, such as at the tip 650 of the feature 670 as shown in FIG. 6A. Sensor 620 may be a glucose oxidase sensor, including a protective membrane, that is hydrated with an aqueous solution or partially aqueous solution. The sensor 620 may be dip coated with a sacrificial layer 680, that can later be removed with in response to heat and/or an aqueous solution with or without a mild detergent to help remove a layer 680 such as wax. In some examples, the sacrificial layer 680 may be a wax layer. At least because the sacrificial layer 680 includes a material, such as wax, that is configured to be removed, efforts may be made to reduce potential damage to sensor 620 from the sacrificial layer 680. For example, sensor 620 may be wetted or coated with trehalose, which will prevent impairment of the sensor 620 during or after removal of the sacrificial layer 680. For example, in embodiments where the sacrificial layer 680 is wax, the removal of the sacrificial layer 680 will not bind to the features of the sensor 620 that are hydrated with water or coated with trehalose, thus avoiding damage to the sensor 620.

With reference to FIG. 6B, where like numerals refer to like features, the sacrificial layer 680 has been removed leaving the sensor 620 coupled to the feature 670. With reference to FIG. 6C, where like numerals refer to like features, the feature 670 is shown with sensors 620, 622 coupled thereto. The sensor 622 may be an aptamer sensor and the sacrificial layer 680 will be removed after fabrication of the aptamer sensor 622.

With reference to FIG. 6D, embodiments of the present invention and in an alternate embodiment to FIG. 6C, no sacrificial layer is required. Rather, an aptamer sensor 622 can be formed from aqueous solutions of alkylthiol blocking molecules and aptamers. The aptamer sensor 622 can be coupled to the feature 670 along the length of the feature 670. Sensor 620, such as a glucose oxidase sensor, may be protected by size-selective membranes such as those already used to protect and diffusion-limit glucose transport in enzymatic sensors, which have small enough pores to block aptamers that have molecular weight of 10's kDa while glucose is only 180 Da, and similarly, such membranes can be hydrophilic to allow diffusion of glucose but not a small hydrophobic molecule such as mercaptohexanol or mercaptooctanol. The membrane could be in the same location as previously described feature 680 but is different by not being removed and sacrificial but rather being a membrane that stays on the glucose sensor 620. Even in the event that some aptamers are able to penetrate the size-selective membranes, alkylthiol and aptamer chemistry need not overly distort the enzymatic sensor's 620 function. Therefore, a sensor 620, such as a glucose sensor, can be fabricated including a protective membrane and then a sensor 622, such as an aptamer sensor, can be fabricated using techniques known to fabrication of aptamer sensors without significant concern of impact on the sensor 620.

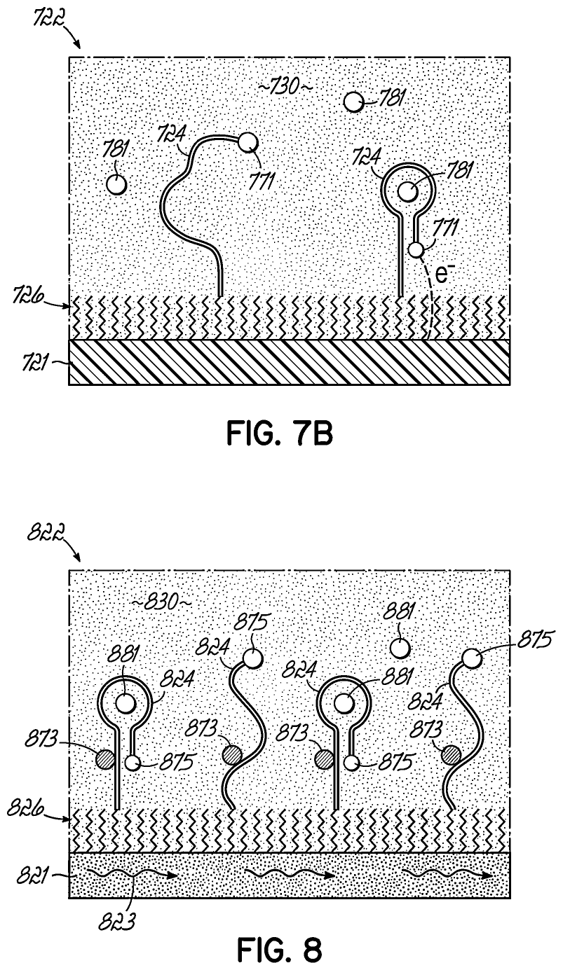

With reference to FIG. 7A, a cross-section of an embodiment the aptamer sensor 222, 322, 422, 522, 622 is shown. Specifically, FIG. 7A shows a cross-section of a redox aptamer sensor 722. The redox aptamer sensor 722 includes at least one substrate 721, which may be an electrode made of a material such as gold, carbon, or other suitable electrode material. The redox aptamer sensor 722 also includes at least one blocking layer 726 which includes a plurality of molecules such as mercaptohexanol, mercaptooctanol, hexanethiol, peptides, or combinations thereof that may be thiol bonded to the substrate 720, or a plurality of natural solutes, some of which may be found in blood for example, that can act as the blocking layer 726, or other suitable molecules. The specific species of molecules included in the blocking layer 726 may depend on the particular application of the sensor 222, 322, 422, 522, 622, and on the choice of substrate 720 material. The redox aptamer sensor 722 further shows at least one aptamer 724 that is responsive to binding to an analyte 781, and which includes a redox tag 771 such as methylene blue. As shown in FIG. 7A, the aptamers 724 a simple stem loop (hairpin) aptamers configured to bond to an analyte 781 found in sample fluid 730. In some embodiments, sample fluid 730 is a biofluid, such as, but not limited to blood, interstitial fluid, sweat, or other biological fluid. Other forms of aptamers are acceptable as well, such as those for vancomycin, phenylalanine, or other analytes that are valuable to measure in the body. FIG. 7A shows the analyte 781 in the sample fluid 730 prior to bonding with the aptamer 724. FIG. 7B shows the redox aptamer sensor 722 after the analyte 781 bonds with the aptamer 724. As shown in FIG. 7B, the bonding of the aptamer 724 to the analyte 781 causes the stem loop to form and the redox current (labelled as “e⁻” in FIG. 7B) measured from the redox tag 771 to increase, as measured using square wave voltammetry or other suitable technique. In an absence of analyte 781 binding to the aptamer 724, the stem loop conformation does not form, and the redox current would thus not increase. Thus, a measurement of electrical current from the redox tag 771 can be used to interpret changes in the amount of analyte 781 bonded to the aptamer 724, and accordingly related to, for example, the concentration of the analyte 781 in the sample fluid 730.

With reference to FIG. 8, where like numerals refer to like features-embodiments of the invention may also use optical aptamer sensors 822, such as a fluorescent optical sensor, where, for example in FIG. 8, a substrate 821 can be a thin glass or polymer (such as acrylic) waveguide propagating excitation light 823 shown by wavy arrows in FIG. 8. The optical aptamer sensors 822, such as a fluorescent optical sensor, may include anti fouling chemistry 826 such as a monolayer of silane attached zwitterions, and aptamers 824 tagged with one or more optical tags, such as quencher tag 873 and fluorescent tag 875. In FIG. 8, shows only an example of specific optical tags that could be used, and is not intended to be a limiting example of all optical aptamer sensors 822 suitable for use in the invention. Optical tags could be a blackhole quencher tag 873 and a fluorescent tag 875 such that when analyte 880 binds to aptamer 824 fluorescence from fluorescent tag 875 is quenched. As used herein, the term “fluorescent tag”, “tag”, and “fluorescent quencher”, and “quencher” means molecules which are like those used in molecular beacon laboratory assays. Examples of fluorescent tags include 6-FAM (carboxylflourescien), JOE, TET, HEX, and examples of quenchers include black-hole quenchers, DABCYL. These tags may also be referred to as “optical tags” more generally, as there are multiple types of optical emission beyond fluorescence such as phosphorescence, and because other optical properties such as optical absorbance magnitude or peak wavelength for optical absorption or fluorescence resonance energy transfer (FRET) can also be measurable aspects of the tags. Because aptamers 824 are within roughly a wavelength of light distance from substrate 821, such as a waveguide, they can be excited by excitation light 823 through evanescent coupling or via light escaping substrate 821, such as a waveguide, through refraction or scattering, and for aptamers 824 that are not bound to analyte 881 they will emit fluorescent light of a longer wavelength than excitation light 823 that can then be coupled back into substrate 821, such as a waveguide, and detected with a photodetector. There are a large number of prior art examples, as taught for example in “Situma C, Moehring A J, Noor M A, Soper S A. Immobilized molecular beacons: a new strategy using UV-activated poly (methyl methacrylate) surfaces to provide large fluorescence sensitivities for reporting on molecular association events. Anal Biochem. 2007 Apr. 1; 363(1):35-45. Doi: 10.1016/j.ab.2006.12.029. Epub 2006 Dec. 20. PMID: 17300739; PMCID: PMC2836515.” Or for example as taught in “De Acha N, Elosúa C, Arregui FJ. Development of an Aptamer Based Luminescent Optical Fiber Sensor for the Continuous Monitoring of Hg²⁺ in Aqueous Media. Sensors (Basel). 2020 Apr. 22; 20(8):2372. Doi: 10.3390/s20082372. PMID: 32331372; PMCID: PMC7219322.” The aptamers generally, but do not always, require at least two tags (fluorescent and quencher tags) and a linker to bind the aptamer to the substrate, and any one of the tags or linkers can be attached at the 3′ end of the aptamer, the 5′ end of the aptamer, or at internal locations where the most common and widely utilized location is at an internal thymine site of the aptamer.

A method of determining a concentration of one or more analytes in a sample fluid is also provided. The method includes providing a first sensor including a plurality of enzymes, the plurality enzymes each including at least one binding site configured to react to the first analyte. The method further including providing a second sensor including a plurality of aptamers, the plurality of aptamers each including at least one binding site configured to bind to the second analyte. The method further including providing the sample fluid including the first analyte and the second analyte. The method further including introducing the sample fluid to the plurality of plurality of enzymes and the plurality or aptamers to allow binding of the first analyte to at least one of the plurality of enzymes and allow binding of the second analyte to at least one of the plurality of aptamers, wherein the first analyte reacting the to at least one of the plurality of enzymes produces a change in a first signal having a first signal strength and the second analyte binding to the at least one of the plurality of aptamers produces a second change in a second signal having a second signal strength. The method further including depleting the first analyte from being unbound to at least one of the plurality of enzymes to the extent that the first signal strength is unchanged for a period of time. The method further including depleting the second analyte from being unbound to at least one of the plurality of aptamers to the extent that the second signal strength is unchanged for a period of time. The method further including calculating the initial concentration of the first analyte in the sample fluid based on the first signal strength after depleting the first analyte from being unreacted to at least one of the plurality of enzymes to the extent that the first signal strength is unchanged for the period of time. The method further including calculating the initial concentration of the second analyte in the sample fluid based on the second signal strength after depleting the second analyte from being unbound to at least one of the plurality of aptamers to the extent that the second signal strength is unchanged for the period of time.

With reference to embodiments of the present invention, electrochemical sensors require reference and counter electrodes. Placing too many electrodes into the dermis of the skin 12 can be challenging. To overcome this obstacle, in embodiments of the present invention, an advantage of using at least two sensors is that the sensor 222, 322, 422, 522, 622 can act as a reference electrode by using a redox tag such as methylene blue that is suitably invariant in its redox potential in blood conditions. A common counter electrode can be situated outside the skin 12 of the user in the form of an adhesive like those used in electrodes for cardiac monitoring and sensors 220, 320, 420, 520, 620, 222, 322, 422, 522, 622 can then share the common counter electrode. Therefore, the present invention may include a first sensor 220, 320, 420, 520, 620 for glucose, a second aptamer sensor 222, 322, 422, 522, 622 that is also a reference electrode for both the aptamer and glucose sensors, and at least one counter electrode that is placed either inside or outside the body but which is in electrical contact with the body.

With reference to embodiments of the present invention, a device may include at least one glucose sensor 220, 320, 420, 520, 620 and at least one aptamer sensor 222, 322, 422, 522, 622 for a small molecule of molecular weight <1000 Da, as will be taught in the examples section.

With reference to embodiments of the present invention, a device may include at least one glucose sensor 220, 320, 420, 520, 620 and at least one aptamer sensor 222, 322, 422, 522, 622 for a small molecule of molecular weight <1000 Da and at least one large molecule sensor of molecular weight >5000 Da, as will be taught in the examples section, with a third or more sensor(s) be added to the device using one or more of the techniques as taught herein.

With reference to embodiments of the present invention, a device may include at least one sensor 220, 320, 420, 520, 620 for a drug and at least one sensor 222, 322, 422, 522, 622 for a molecule or biomarker that is regulated by the drug, as will be taught in the examples section.

### EXAMPLES

### Example 1

A device includes a first sensor for glucose and a second aptamer sensor for a drug molecule such as a blood thinner such as rivaroxaban or warfarin or apixaban. An example rivaroxaban aptamer sequence that enables such a sensor is 5′-GGA CGA CAC CGC TGC GAT ACG GTG ATA CAA TTG TAC CGC ACT GGA TTG TCG T-3′ [SEQ ID NO: 1] and is fabricated as follows with thiol termination and methylene blue termination of the aptamer. First, a pristine gold surface capable of binding thiolated molecules is prepared by any combination of mechanical, thermal, chemical, or electrochemical methods including but not limited to those currently employed in the art of aptamer-based sensor fabrication. An example protocol includes mechanical polishing of the gold surface with either diamond or alumina slurry, followed by sequential electrochemical cycling in basic and acidic solutions. Second, the gold electrode is immersed either sequentially or simultaneously in a solution containing the thiolated aptamer sequence modified with a redox tag and a solution containing molecules suitable for a blocking layer, an example of which is mercaptohexanol. Mixed monolayer formation may occur through self-assembly or through electrochemical assistance to tune sensor performance. Incorporation of the sensor for glucose may be achieved prior to, concomitantly, or proceeding fabrication of the aptamer sensor by means of, but not limited to, any of the aforementioned methods. Given the small molecular weight of most blood thinners, such as rivaroxaban (436 Da), a size-selective encapsulating membrane or coating may be applied to the aptamer sensor to prevent biofouling and degradation via large endogenous molecules, such as albumin, while still allowing for access of analyte to the aptamer sensor.

Glucose sensors last for 10-14 days of use, and a blood thinner sensor may, but need not, operate that long. For example, a device may include a glucose sensor that at lasts for least 7 days, and a rivaroxaban sensor that lasts for at least one of 1 day, 2 days, 3 days, or even at least 5 days. This is sufficient to periodically provide a user with concentration profiles over the course of multiple dosing intervals given a typical rivaroxaban dosing regimen of twice daily, allowing for dosing optimization of rivaroxaban that can be achieved at least at the onset of each placement of such a device every 10-14 days. Measured unbound concentrations in blood may range from 10's to 100's of nM.

### Example 2

A device includes a first sensor for glucose and a second aptamer sensor for a molecule such as a blood thinner such as rivaroxaban and a third aptamer sensor for a biomarker such as thrombin or troponin or brain-natriuretic peptide. The second sensor alternately could be for the biomarker and no third sensor added. A thrombin sensor may be fabricated using an aptamer sequence of 5′-TAA GTT CAT CTC CCC GGT TGG TGT GGT TGG T-3′ [SEQ ID NO: 2]. A thrombin sensor can measure the decrease in thrombin generation caused by dosing of rivaroxaban or apixaban. Alternately an aptamer can be selected for measuring in-activated vs. in-activated thrombin by selecting for the aptamer with and without the presence of a blood thinner that inhibits thrombin affinity. Alternately a troponin sensor may be fabricated using an aptamer sequence of AGTCTCCGCTGTCCTCCCGATGCACTTGACGTATGTCTCACTTTCTTTTCATTGAC ATGGGATGACGCCGTGACTG [SEQ ID NO: 3]. The aptamer sensors in such a device may be prepared individually on separate electrodes as described in Example 1, placed on the same or on an adjacent substrate. A size-selective encapsulating membrane or coating may be applied to the rivaroxaban aptamer sensor to prevent biofouling and degradation via large endogenous molecules, such as albumin, while still allowing for access of analyte to the aptamer sensor. Since thrombin or pro-thrombin are large molecules (˜36 kDa and ˜72 kDa respectively), a size-selective membrane would not preclude the access of large endogenous molecules (>5000 Da) to the sensor that may cause sensor degradation. In this regard, chemical or electrochemical modifications to the sensor may be realized in efforts to thwart sensor degradation. Another realization may include the functionalization of a single working electrode with both aptamer sequences, each possessing a redox tag with a redox potential distinguishable from the other such that currents and concentration estimates can be ascertained from interrogation of a singular working electrode.

Glucose sensors last for 10-14 days of use, and a blood thinner sensor may, but need not, operate that long. For example, a device may include a glucose sensor that lasts for least 7 days, and a rivaroxaban sensor that lasts for at least, in alternate embodiments, 1 day, 2 days, 3 days, or even at least 5 days and a thrombin sensor that lasts for at least 1 day. Thrombin is large at ˜36 k Da, and therefore more difficult to make a long-lasting sensor for because it is difficult to protect the sensor from fouling by other large solutes in the biofluid such as albumin. This approach is sufficient to periodically provide a user of concentration profiles for optimizing dosing of rivaroxaban for at least 1 day at the onset of device incorporation.

### Example 3

A device includes a first sensor for glucose and a second aptamer sensor for an anti-inflammatory drug such as a corticosteroid steroid and a third sensor for C-Reactive Protein using an aptamer sequence such as

[SEQ ID NO: 4]
GCCTGTAAGGTGGTCGGTGTGGCGAGTGTGTTAGGAGAGATTGC.

Corticosteroid therapy can lower C-Reactive protein by 50% in several days after starting therapy. C-Reactive Protein can be measured continuously but in many applications its longitudinal measurement is more valuable. Therefore single reading of C-Reactive Protein taken from one measurement or even 1 day or more of measurements may be sufficient for a device that is repeatedly worn (e.g. with a 10-day glucose meter a C-Reactive protein measure would be provided every 10 days when the user applies the device).

### Example 4

A device includes a first sensor for glucose and a second aptamer sensor for insulin. The insulin can be measured continuously and/or just for a shorter period such as hours or days to help track insulin resistance or auto-immune issues with insulin or to help the accuracy of a glucose sensor for managing a patients diabetes. An example insulin aptamer that can be used is AAAAGGTGGTGGGGGGGGTTGGTAGGGTGTCTTCT [SEQ ID NO: 5] and to achieve limits of detection requirement a second aptamer can be used to achieve bivalent binding (two binding sites) where the aptamers are connected by a flexible linker.

### Example 5

A device includes a first sensor for glucose and a second aptamer sensor for BNP or NT-proBNP. The NT-proBNP can be measured continuously and/or just for a shorter period such as hours or days to help track heart failure status. An example NT-proBNP aptamer that can be is used

[SEQ ID NO: 6]
GGCAGGAAGACAAACAGGTCGTAGTGGAAACTGTCCACCGTAGACCGGT
TATCTAGTGGTCTGTGGTGCTGT.

### Example 6

A device includes a first sensor for glucose, at least one aptamer sensor, and a third sensor for creatinine for kidney function or other disease states. An example enzymatic creatinine sensor is comprised of two sub sensors using sarcosine oxidase, creatine amidinohydrolase and creatinine amidohydrolase for three-stage enzymatic catalysis of creatinine to electrochemically detectable hydrogen peroxide (H2O2). Two subsensors are usually required for the subtractive determination of creatinine from a combination of creatine and creatinine. The concentration of creatinine and creatine is determined using all three enzymes, whereas a second sensor that omits creatinine amidohydrolase, measures creatine. Thus, the creatinine concentration can be easily determined. The concentration of either creatine or creatine and creatinine is proportional to the amount of H2O2 generated, which is detected by reduction at the surface of an electrode. A creatinine sensor can also be potentiometric using catalysis of creatinine by creatinine iminohydro-lase (CIH) at the surface of an NH4-sensing ion-selective electrode. Alternately, a creatinine aptamer sensor can be used by applying commercially available creatinine aptamers that can be purchased from Creative Biolabs—Anti-Creatinine Aptamer (Cat #: CTApt-865).

### Example 7

A device includes a first sensor for glucose and a second aptamer sensor for potassium. The potassium can be measured continuously and/or just for a shorter period such as hours or days to help track heart failure or kidney failure status or other diseases or health. Example aptamer sequences that can be attached via silane (on oxides) or thiol chemistry (on golds) and be tagged with methylene blue at the opposite end of the aptamer include but are not limited to the following and can be further modified to tune their binding affinity and linear range:

[SEQ ID NO: 7]
5′-CCAACGGTTGGTGTGGTTGG-3′
[SEQ ID NO: 8]
5′-CCAAGGTTGGTGTGGTTGG-3′
[SEQ ID NO: 9]
5′-ACCAAGGTTGGTGTGGTTGG-3′
[SEQ ID NO: 10]
5′-CCCAAGGTTGGTGTGGTTGG-3′
[SEQ ID NO: 11]
5′-CAAGGTTGGTGTGGTTGG-3′
[SEQ ID NO: 12]
5′-GGTTGGTGTGGTTGG-3′
[SEQ ID NO: 13]
5′-AAAATGAGGGAGGGG-3′
[SEQ ID NO: 14]
5′-AAAATGGACAAACGA-3′
[SEQ ID NO: 15]
5′-AAAAGGGTTAGGGTTAGGGTTAGGGAAAAGCGTCCTCCG-3′
[SEQ ID NO: 16]
5′-AAAACGGAGGACGC-3′
[SEQ ID NO: 17]
5′-AAAAGGGTTAGGGTTAGGGTTAGGG-3′.

### Example 8

Sensing devices such as those taught herein often require membrane protection but membrane protection for glucose sensors are too tight for most analytes to be measured by aptamers. Glucose is only 180 Da in molecular weight, and in glucose sensors the membrane is purposely diffusion limiting to work well, and so diffusion limiting that it could cause very long lag times (10's to 100's minutes) for aptamers and in some cases such as protein sensing preclude sensing at all (CRP is an excellent example at 120,000 Da). Glucose protecting membranes are typically at least in part solution coated onto both the sensor and substrate such as 220 and 270 and include polycarbonate and cellulose acetate, polyurethane compositions capable of absorbing from 10 to 50% of their dry weight of water, nafion, polyvinylpyridine. The present invention therefore may include first coating an embodiment such as device 200 only with a first glucose-flux-limiting membrane over sensor 220, curing said membrane, and then coating the rest of the device 200 with a membrane required for the aptamer sensor such as 222. An exemplary membrane is polybetaine and can be formed as follows: zwitterionic polybetaine-based hydrogel can be dip coated from 1 μL of the aqueous mixture consisting of monomer/cross-linker/photo-initiator (2.8 g/1.8 μl/36 μl respectively dissolved in 1 ml of DI water) over the aptamer sensor and exposing it to UV light (λ:280-450 nm, Bluewave LEDPrime UVA, Dynamax, USA) for 45 min. In some cases wetting agents or viscosity increasing agents can add to the thickness of the membrane, even include for example a suspension of nanocellulose or other filler. Subsequently, the polysulfobetaine-coated sensors are immersed in 1×PBS to remove any left over mononomer then can be dried with a sensor preservative such as trehalose. The above example will work for up to analytes of several kDa or larger and a less dense membrane can be used for even larger analytes. As a result the present invention may include a first membrane coated onto the glucose sensor and a second membrane coated onto both the glucose and aptamer sensors where the second membrane is at least 5× more permeable to glucose than the first membrane.

### Example 9

With reference to FIGS. 7A-7B, an example redox aptamer sensor 722 as placed initially in a sample fluid 730, such as interstitial fluid, is shown. The redox aptamer sensor 722 includes at least one substrate 721, such as a working electrode made of a material such as gold, carbon, or other suitable electrode material; at least one protective layer that is a monolayer protective layer 726 such as a plurality of molecules such as mercaptohexanol or mercaptooctanol that are thiol bonded to the electrode; at least one aptamer 724 that is responsive to binding to an analyte 781; and a redox tag 771, such as methylene blue, associated with the at least one aptamer, such as by being bound thereto. In the generic example shown in FIGS. 7A and 7B, the aptamer 724 is a simple stem loop (hairpin) aptamer where analyte 781 binding causes the stem loop to form and the redox tag current measured from the redox tag 771 to increase, as measured using square wave voltammetry, chronoamperometry, or other suitable technique. In absence of analyte 781 binding to the aptamer 724 the stem loop conformation does not form and the redox current thus does not increase. Alternately, analyte 781 binding to different example aptamer can cause a decrease in redox tag current. Thus, with aptamer sensors changes in a measurement of electrical redox tag current can be used as a signal to interpret changes in concentration of the analyte 781. A non-limiting specific example use case and chemistries are given in “Watkins Z, Karajic A, Young T, White R, Heikenfeld J. Week-Long Operation of Electrochemical Aptamer Sensors: New Insights into Self-Assembled Monolayer Degradation Mechanisms and Solutions for Stability in Serum at Body Temperature. ACS Sensors [Internet]. 2023 Mar. 8; Available from: https://doi.org/10.1021/acssensors.2c02403”.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

### REFERENCE TO AN ELECTRONIC SEQUENCE LISTING

The contents of the electronic sequence listing (UOC22096WO.xml; Size: 14 kilobytes; and Date of Creation: Apr. 19, 2023) is herein incorporated by reference in its entirety.

What is claimed is:

1. A wearable device for sensing at least one analyte in a biofluid, the wearable device comprising:
at least one feature configured to penetrate a skin of a user;
a first sensor coupled to the feature, the first sensor configured to detect a first analyte, wherein the first sensor is in fluid communication with the feature; and
a second sensor comprising a plurality of aptamers configured to detect a second analyte, the second analyte being an analyte different from the first analyte, wherein the second analyte is in fluid communication with the feature.

2. The device of claim 1 wherein the feature comprises a needle.

3. The device of claim 1, wherein the feature comprises Kapton film, gold or stainless-steel.

4. The device of claim 1, wherein at least one of the first sensor or the second sensor is an enzymatic sensor.

5. The device of claim 1, wherein the first analyte is glucose.

6. The device of claim 1, wherein the first sensor or the second sensor is an aptamer-based sensor.

7. The device of claim 1, wherein the first sensor is a continuous monitoring sensor configured to measure the first analyte during a use of the wearable device.

8. The device of claim 7, wherein the second sensor is a continuous monitoring sensor configured to measure the second analyte during the use of the wearable device.

9. The device of claim 7, wherein the second sensor is a non-continuous monitoring sensor configured to measure the second analyte during only a portion of the use of the wearable device.

10. The device of claim 9, wherein the second sensor is configured to provide a single concentration reading of the second analyte.

11. The device of claim 1, wherein the second analyte is a drug.

12. The device of claim 11, further comprising a third sensor wherein the third sensor is configured to detect an analyte.

13. The device of claim 1, wherein the second analyte is a biomarker analyte.

14. The device of claim 13, further comprising a third sensor wherein the third sensor is configured to detect a second biomarker analyte that is not the same biomarker analyte that is configured to be detected by the second sensor.

15. The device of claim 1, wherein at least one of the first analyte or the second analyte is a blood thinner drug.

16. The device of claim 1, wherein at least one of the first analyte or the second analyte is a c-reactive protein.

17. The device of claim 1, wherein at least one of the first analyte or the second analyte is a troponin.

18. The device of claim 1, wherein at least one of the first analyte or the second analyte is a brain-natriuretic peptide.

19. The device of claim 1, wherein at least one of the first analyte or the second analyte is insulin.

20. The device of claim 1, wherein the first sensor is an enzymatic sensor and is covered by a first membrane, and the second sensor is an aptamer sensor, and the first and the second sensors are both further covered by a second membrane that is at least 5× more permeable to glucose than the first membrane.

21. A method of determining an initial concentration of a first analyte and a second analyte in a sample fluid, the method comprising:
providing a first sensor including a plurality of enzymes, the plurality enzymes each including at least one binding site configured to react to the first analyte;
providing a second sensor including a plurality of aptamers, the plurality of aptamers each including at least one binding site configured to bind to the second analyte;
providing the sample fluid including the first analyte and the second analyte;
introducing the sample fluid to the plurality of plurality of enzymes and the plurality or aptamers to allow binding of the first analyte to at least one of the plurality of enzymes and allow binding of the second analyte to at least one of the plurality of aptamers, wherein the first analyte reacting the to at least one of the plurality of enzymes produces a change in a first signal having a first signal strength and the second analyte binding to the at least one of the plurality of aptamers produces a second change in a second signal having a second signal strength;
depleting the first analyte from being unbound to at least one of the plurality of enzymes to the extent that the first signal strength is unchanged for a period of time;
depleting the second analyte from being unbound to at least one of the plurality of aptamers to the extent that the second signal strength is unchanged for a period of time;
calculating the initial concentration of the first analyte in the sample fluid based on the first signal strength after depleting the first analyte from being unreacted to at least one of the plurality of enzymes to the extent that the first signal strength is unchanged for the period of time; and
calculating the initial concentration of the second analyte in the sample fluid based on the second signal strength after depleting the second analyte from being unbound to at least one of the plurality of aptamers to the extent that the second signal strength is unchanged for the period of time.

22. A wearable device for sensing at least one analyte in a biofluid, the wearable device comprising:
at least one feature configured to penetrate a skin of a user;
a sensor coupled to the feature, the sensor configured to detect the at least one analyte, wherein the sensor is in fluid communication with the feature; and
a sacrificial layer housing the sensor, the sacrificial layer being removable from the wearable device in response to at least one of an application of a predetermined amount of heat or solution to dissolve the sacrificial layer.


SEQUENCE LISTING (17 sequences)

SEQ ID NO: 1 - DNA, 52 bp, synthetic construct
  source 1..52: mol_type = other DNA
ggacgacaccgctgcgatacggtgatacaattgtaccgcactggattgtcgt

SEQ ID NO: 2 - DNA, 31 bp, synthetic construct
  source 1..31: mol_type = other DNA
taagttcatctccccggttggtgtggttggt

SEQ ID NO: 3 - DNA, 76 bp, synthetic construct
  source 1..76: mol_type = other DNA
agtctccgctgtcctcccgatgcacttgacgtatgtctcactttcttttcattgacatgggatgacgccgtgactg

SEQ ID NO: 4 - DNA, 44 bp, synthetic construct
  source 1..44: mol_type = other DNA
gcctgtaaggtggtcggtgtggcgagtgtgttaggagagattgc

SEQ ID NO: 5 - DNA, 35 bp, synthetic construct
  source 1..35: mol_type = other DNA
aaaaggtggtggggggggttggtagggtgtcttct

SEQ ID NO: 6 - DNA, 72 bp, synthetic construct
  source 1..72: mol_type = other DNA
ggcaggaagacaaacaggtcgtagtggaaactgtccaccgtagaccggttatctagtggtctgtggtgctgt

SEQ ID NO: 7 - DNA, 20 bp, synthetic construct
  source 1..20: mol_type = other DNA
ccaacggttggtgtggttgg

SEQ ID NO: 8 - DNA, 19 bp, synthetic construct
  source 1..19: mol_type = other DNA
ccaaggttggtgtggttgg

SEQ ID NO: 9 - DNA, 20 bp, synthetic construct
  source 1..20: mol_type = other DNA
accaaggttggtgtggttgg

SEQ ID NO: 10 - DNA, 20 bp, synthetic construct
  source 1..20: mol_type = other DNA
cccaaggttggtgtggttgg

SEQ ID NO: 11 - DNA, 18 bp, synthetic construct
  source 1..18: mol_type = other DNA
caaggttggtgtggttgg

SEQ ID NO: 12 - DNA, 15 bp, synthetic construct
  source 1..15: mol_type = other DNA
ggttggtgtggttgg

SEQ ID NO: 13 - DNA, 15 bp, synthetic construct
  source 1..15: mol_type = other DNA
aaaatgagggagggg

SEQ ID NO: 14 - DNA, 15 bp, synthetic construct
  source 1..15: mol_type = other DNA
aaaatggacaaacga

SEQ ID NO: 15 - DNA, 39 bp, synthetic construct
  source 1..39: mol_type = other DNA
aaaagggttagggttagggttagggaaaagcgtcctccg

SEQ ID NO: 16 - DNA, 14 bp, synthetic construct
  source 1..14: mol_type = other DNA
aaaacggaggacgc

SEQ ID NO: 17 - DNA, 25 bp, synthetic construct
  source 1..25: mol_type = other DNA
aaaagggttagggttagggttaggg